(19) United States
(12) Patent Application Publication
(10) Pub. No.: US 2017/0303802 A1
(43) Pub. Date: Oct. 26, 2017

(54) BLOOD PRESSURE METER AND LESS INDIVIDUAL DEPENDENT CUFF THEREOF

(71) Applicant: NEC Corporation, Minato-ku, Tokyo (JP)

(72) Inventors: Ersin ALTINTAS, Tokyo (JP); Masahiro KUBO, Tokyo (JP); Katsumi ABE, Tokyo (JP); Kimiyasu TAKOH, Tokyo (JP); Yuji OHNO, Tokyo (JP); Takeshi AKAGAWA, Tokyo (JP); Tetsuri ARIYAMA, Tokyo (JP)

(73) Assignee: NEC Corporation, Minato-ku, Tokyo (JP)

(21) Appl. No.: 15/517,299

(22) PCT Filed: Oct. 10, 2014
(86) PCT No.: PCT/JP2014/077762

§ 371 Date: Apr. 6, 2017

(51) Int. Cl. A61B 5/022; A61B 5/021

(52) U.S. Cl. CPC A61B 5/02233; A61B 5/02108

(57) ABSTRACT

A blood pressure cuff less dependent on individual's arm shape with downsized dimensions is introduced with medical approval accuracy. The present invention comprises an occlusion component configured to occlude the artery, and a flexible plastic core to limit the degree of freedom of said occlusion component towards body portion, and a spacer occupying the volume between said core and said occlusion component to improve the compliance towards body portion.

### TECHNICAL FIELD

This invention is related to a blood pressure meter and cuff.

### BACKGROUND ART

Blood pressure is one of the vital signs (i.e. blood pressure, breathe, temperature, heart pulse etc.) in humans or animals, and it is one of the strongest parameters to monitor and to diagnose the medical conditions and the diseases such as heart diseases and hypertension. For a reliable medical evaluation and treatment, blood pressure measurement accuracy less than ±5 mmHg with a deviation within ±8 mmHg is necessary from a body portion. Since, the blood pressure value is strongly dependent on the vertical distance from the heart level; blood pressure measurement from upper arm at the level of heart is universally recognized by medical professionals for a more reliable and accurate measurement. This is generally achieved by a structure called “cuff”, which is wrapped (or placed) around upper arm of human.

Usual cuffs are composed of bags or bladders inflated/deflated (or pressurized/depressurized) by air through a pressure control unit. In order to measure the blood pressure, there can be different methods such as (i) detection of Korotkoff sounds usually achieved by a stethoscope by medical professionals, (ii) oscillometric techniques detecting the oscillations in the inflatable air bag due to pressure oscillations caused by artery, and (iii) techniques depending on Doppler Effect. Korotkoff sounds and oscillometric detections are widely accepted and employed in commercial blood pressure monitors, meters or devices (i.e. sphygmomanometer). In the case of automatic or electronic blood pressure meter, oscillometric methods are usually employed due to its improved signal to noise ratio capabilities and no need of detection of Korotkoff (blood flow) sounds. Furthermore, this method allows visualizing blood pressure wave or pulse wave, and it improves the medical evaluation of a subject.

During the pressurization of the cuff, however, the heart continues to pump the blood and it hits to the walls of the occluded artery under the cuff. The blood flow from the heart side reflects back and causes upstream flows in the proximal side. The cuff under the pressurization resembles an ellipsoid in cross-section, and it loses the efficiency of the contact with skin at the edges. This is known as cuff-edge effect. It causes a non-uniform pressure distribution over the artery leading to a partial occlusion or a narrower occlusion of the artery around the center of the cuff. Due to cuff-edge effects, the effective occlusion width is smaller than that of the cuff along the axis around which the cuff is wrapped.

The cuff size for an upper arm type blood pressure monitor is an important consideration. The ideal cuff should have a bag width at least 40% of the arm circumference, and double of the width is recommended for the length of the bag. For a small adult with an arm circumference of 22 to 26 cm, 12 cm bag width is recommended, while for a standard adult with an arm circumference of 27 to 34 cm (or more), 16 cm bag width is recommended [NPL (Non Patent Literature) 1, page 705]. However, these considerations are probably based on cuffs composed of single air bag (bladder) suffering from cuff-edge problems.

A cuff with a bag having 12 cm width is used in most of the medical checks. These checks are usually fast and less than 5 minutes. Even though, comfortability is not an issue during medical checks, a cuff width around 12 cm is not comfortable for daily uses and/or for continuous blood pressure measurements, i.e. ambulatory blood pressure measurement (ABPM). It is a known fact that blood pressure measurement results can be affected by white-coat hypertension and cause erroneous results and treatments. The blood pressure measurements out of hospitals, at homes or during daily life are recommended for more reliable results especially to predict the risks of cardiovascular events and to diagnose the white-coat hypertension [NPL 1]. However, current cuffs have large width and they are stressful to the user during daily life. A smaller cuff width without sacrificing the accuracy is appreciated for daily life measurements and it remains as a problem.

Mercury type upper arm blood pressure monitor has been accepted as a gold standard [NPL 1]. Typical commercially available cuff of mercury type blood pressure monitor has an inflatable/deflatable air bag (or occlusion component) width around 12 cm, and its cross section on a body portion (e.g. an arm or leg) of human (or animal) is similar to ellipsoid. The proximal side (near to the heart) is called as upstream side and the distal side (near to the hand or foot) is called as downstream side. The occlusion component is pressurized to occlude the artery and the blood pressure is measured based on the oscillations caused by oscillations in the underlying artery.

Although the aforementioned width with its pressure distribution characteristic in typical cuffs is tolerable, decreasing the width enhances cuff-edge effect, and this will probably cause erroneously high readings [NPL 2] due to probably incompletely and/or non-uniformly transmitted pressure to the artery under a narrower cuff or mis-cuffing. Therefore, if the pressure can be completely or uniformly transmitted to or distributed over the artery under the cuff by reducing those cuff-edge effects, smaller cuff width for standard adults is realizable and applicable with enough measurement accuracy.

In addition to these, fitting the cuff to the body portion or the compliance of the cuff towards body portion is another important consideration. Unfitted cuffs cause erroneous results due to improper occlusion of the underlying artery. The cross section or the shape of the body portion changes from individual to individual. Some may have fatty body portion, while some others can have muscular body portions.

The usual cuffs are made of inflatable bags or bladders. To improve the compliance of the cuff, some flexible and hard structures or cores made by plastics are employed (PLT (Patent Literature) 1-3). For example, in one invention holes in the core are employed to improve the fitness of the cuff the body portion (PLT 1). Relatively hard plastic sheets probably reduce the cuff-edge problem by limiting the motion of the inflatable cuff towards body portion, and therefore improve the sensitivity.

However, in all these inventions (PLT 1-3) inflatable bags are usual in size, and their cuff-edge problems are tolerable. They are still as wide as 14 cm with textile which poses stress to the users or patients in daily life uses such as ABPM. They can decrease the width of the cuff and the inflatable bag to reduce the stress and they can provide fitness of the cuff the body portion to some extent, but this time cuff-edge problems will enhance and medical accuracy will be lost.

A cuff with high fitness to the body portion or less individual dependency (i.e. arm shape independent) with downsized structure (i.e. reduced stress, high wearability and portability) within medical accuracy for ABPM applications remains as a problem.

### CITATION LIST

### Patent Literature

[PLT 1] JP 2003-210423

[PLT 2] U.S. Pat. No. 8,771,196 B2

[PLT 3] JP 2002-209858

### Non Patent Literature

[NPL 1] Thomas G. Pickering et al., “Recommendations for blood pressure measurement in humans and experimental animals. Part 1: Blood pressure measurement in humans: A statement for professionals from the subcommittee of professional and public education of the American Heart Association council of high blood pressure research”, Circulation, 111, 697-716, 2005

[NPL 2] M. Ramsey, “Blood pressure monitoring: Automated oscillometric devices”, J. Clin. Monit., 7, 56-67, 1991

### SUMMARY OF INVENTION

### Technical Problem

Individuals have different arm shapes leading to different upper arm cross-sections. These differences can cause erroneous results of blood pressure due to the fact that the blood pressure cuff does not fit very well or compliance towards body portion is not sufficient. A blood pressure cuff which is less dependent on the individual's arm shape or arm cross-section is appreciated.

### Solution to Problem

A flexible spacer occupying the volume for unfitted space and enhancing the compliance towards body portion is utilized.

### Advantageous Effects of Invention

Although it is downsized, the blood pressure cuff employing a spacer in the present invention achieves medically more accurate and less erroneous blood pressure readings with similar medical accuracies to its commercial counterparts (12 cm bag width). Sensitivity is improved around 25%, and errors or deviations are reduced approximately 40% when a spacer is utilized. The invented downsized cuff with enough accuracy has great potentials of more comfortable, more wearable and more portable medical devices and ABPM applications.

### BRIEF DESCRIPTION OF DRAWINGS

FIG. 1 Measurement results of human upper arm and elliptical approximation to the cross-section of upper arm;

FIG. 2 Cross-section of wrapping and place of spacing;

FIG. 3 Spacer position and preferred shape;

FIG. 4 Different combinations of the first exemplary embodiment of the invention;

FIG. 5A Measurement results before experiments;

FIG. 5B Measurement results after experiments;

FIG. 5C Experimental results and impact of the invention;

FIG. 6 An example of the second exemplary embodiment of the invention with embedded spacer processed in the core;

FIG. 7 Another example of the second exemplary embodiment of the invention with a secondary core attached to the core;

FIG. 8 Example of the third exemplary embodiment of the invention with an elliptically processed core in cross-section along wrapping direction;

### DESCRIPTION OF EMBODIMENTS

Exemplary embodiments for carrying out the present invention will be described with the help of figures in the following. However, although exemplary technical limitations for carrying out the present invention are applied to the exemplary embodiments described below, the scope of the invention is not limited to below.

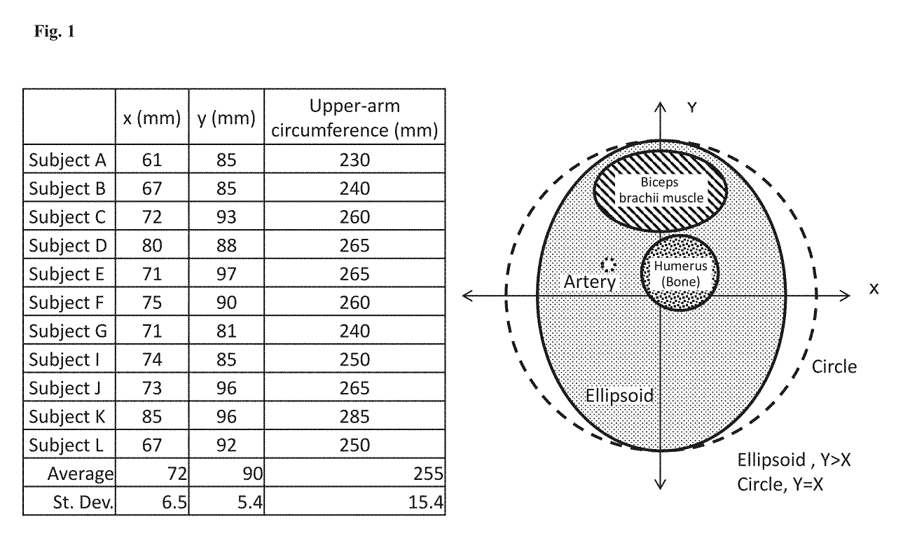

Human upper arm can have different shapes and so different cross sections. Some may have fatty structures while some may have muscular structures. To understand this effectively, we investigated cross-section of human upper arm in 11 subjects (FIG. 1). For example, subject D and K have relatively circular cross-sectioned upper arm while others are very close to the elliptical cross-section.

However, the problem is that the core (flexible plastic sheet which is relatively hard) to improve the fitness are in circular in cross-section. Since, it is quite simple to shape the core in circle without extra cost. PLT 1, 2 and 3 actually utilize such cores. PLT 1 being different from the others employs truncated cone like core while others uses cylindrical like cores. Even though the shapes can be different, all cores are circular in cross section in wrapping direction towards body portion. Since, shaping into circle is quite simple without extra cost.

Processing into elliptical cross section is possible to, but it increases the manufacturing cost which hinders the availability of the device on large populations. Therefore, it is appreciated to have low-cost approaches.

The average cross-section of human upper arm is not circular in cross-section, and it is the best to approximate it as elliptical. On the right sketch of FIG. 1, elliptical cross-section of upper arm is illustrated with biceps brachii muscle at top (Y axis), and humerus bone under of it (left upper arm cross section looked from left hand). On the left of the bone, artery is illustrated. Average arm is approximated or modeled as elliptical in cross-section.

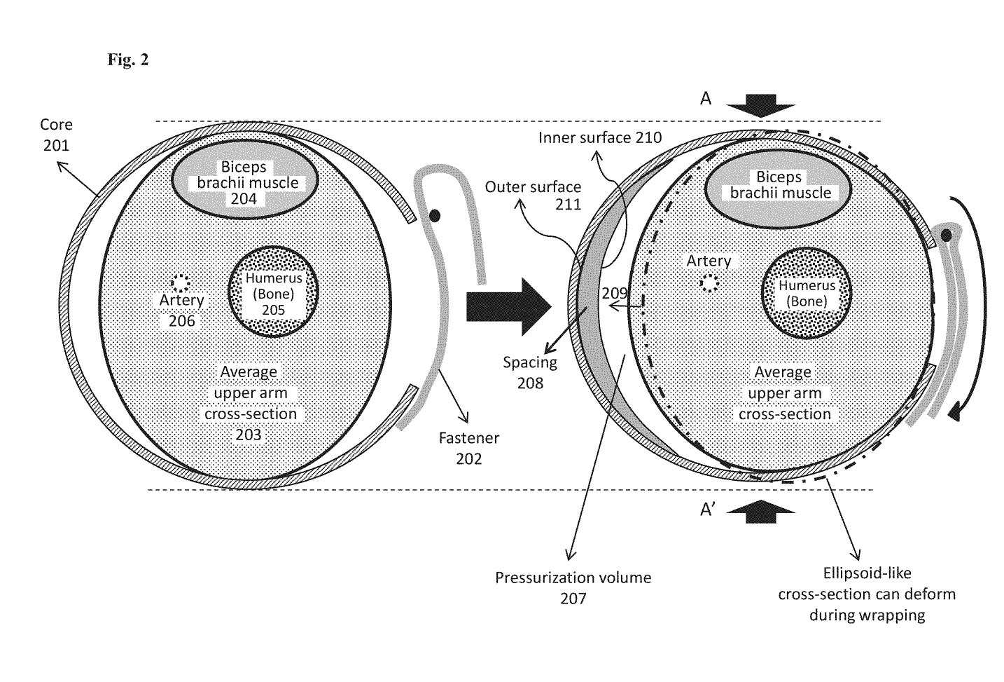

A core 201 of blood pressure cuff is attached to illustrate the condition in body portion, e.g. arm 203 (FIG. 2). Left-hand side figure shows pre-attachment of the core to a body portion modeled elliptical cross-section (average upper arm cross-section). The sketch shows the cross section when someone looks from left hand to left upper arm. Biceps brachii muscle 204 is illustrated at top, and humerus bone 205 at below of it. Artery is roughly left of the humerus bone 205. Thanks to this bone 205 (due to its inflexibility), it is possible to occlude the artery and to measure blood pressure. Fastener 202 is used to wrap the core and to improve the fitness or the compliance of the hard cuff to body portion.

When fastened, the upper arm is generally deformed due to soft body tissues. Forces on the right hand side of AA′ directions are usually responsible for these. This further causes elongation of soft body tissues in 209 directions. Due to the cross-section of the upper arm similar to ellipsoid, and circular cross section of the core, there will be a dead space or unfitted spacing 208 to the body portion. Between body portion and the core 201, there will exist as pressurization volume 207 usually occupied by inflatable bags. This spacing 208 is similar to a crescent in cross-section. It is such that the inner surface 210 of the spacing near to the body portion is smaller than the outer surface 211 near to the core.

This spacing causes insufficient fitness or compliance to the body portion. But, current technologies have large inflatable cuff widths, and errors are tolerable. When downsized, those spaces are critical in importance.

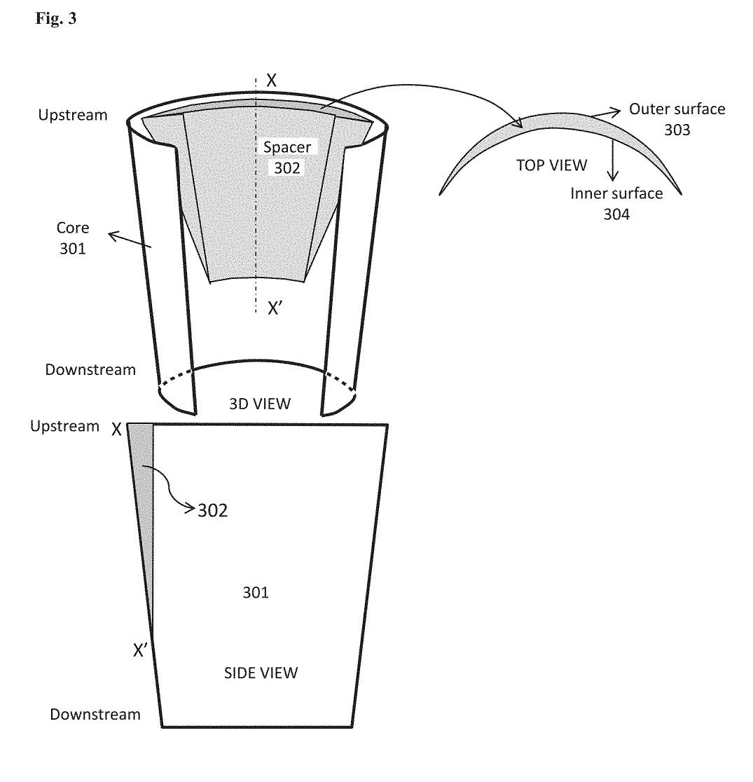

FIG. 3 shows a spacer occupying mentioned spacing above. Core 301 is usually made by flexible plastics with/without holes inside. It is possible to have truncated cone like C-shaped or cylindrical like C-shaped cores. To improve the fitness, truncated cone like C-shaped core is preferable.

Spacer 302 with a comparable or less elasticity relative to the core 301 material is preferable. Metals, plastics (including pored or foamed plastics too) or composites are possible. The spacer surface near to the core is called as outer surface 303, and the spacer surface near to the body portion is called as inner surface 304. The top view of the spacer looks like crescent. Therefore, it is appreciated that inner surface is smaller than outer surface during attachment to the body portion.

In FIG. 3, cross-section of spacer 302 in XX′ direction is shown too. It is such that the distance of inner surface to the outer surface at upstream side is bigger than the distance at downstream side. In the figure, a triangle like structure is illustrated, but other cross-sections are possible too. For example, inner surface at XX′ direction can be curvy.

Another point is that the size of the spacer 302 is equal to or smaller than the size of the core 301. The width in XX′ direction can be equal to or smaller than the width of the core 301. The length of the spacer 302 can be equal to or smaller than the core 301.

### First Exemplary Embodiment

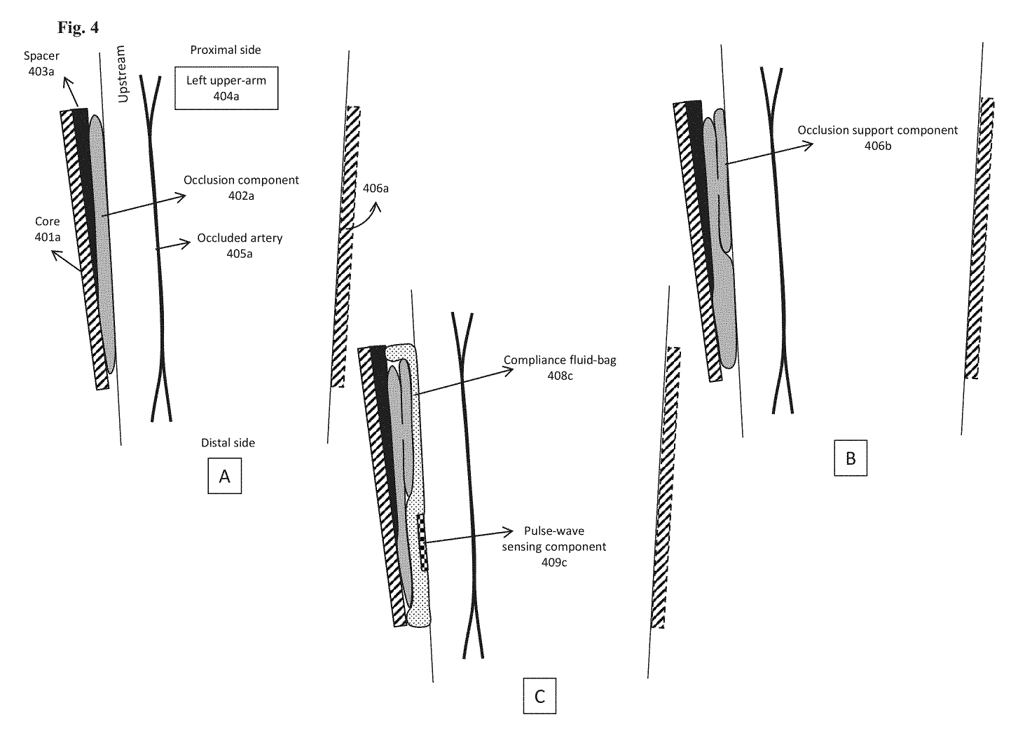

The blood pressure cuff of the first exemplary embodiment is shown in FIG. 4 with 3 different combinations in A, B and C. FIG. 4-A shows that spacer is between occlusion component and the core as the first example of the first exemplary embodiment. FIG. 4-B shows that occlusion component is supported by occlusion support component at upstreams side as the second example of the first exemplary embodiment. FIG. 4-C shows that a compliance fluid bag is attached towards body portion to improve the compliance and a pulse wave detection component is configured between body portion and compliance fluid bag as the third example of the first exemplary embodiment.

FIG. 4-A shows the first example. The blood pressure cuff is depicted to be placed around left upper arm 404a. Top side is near to the heart and called as proximal side, where the opposite side near to the hand is called as distal side. A truncated cone like C-shaped core 401a is preferred. It is fact that this shape is the best fitted structure to upper arm in average. Since, upper arm is mostly thinner in distal side.

Core 401a is preferably a flexible plastic. To occlude the artery, occlusion component 402a is utilized. An inflatable/deflatable air bag is preferable. When occluded component is active, it causes to artery to be occluded (i.e. occluded artery 405a). Occlusion component 402a is the closest component to the body portion. Between core 401a and occlusion component 402a there is a spacer 403a to occupy the space to increase the fitness of the blood pressure cuff (or the compliance) to the body portion. When looked from proximal side (or upstream side) a crescent like cross section is preferred (as depicted in FIG. 3). Since, as mentioned before, crescent like flexible structure is the best to fit a circular truncated cone like C-shaped core to an elliptical cross-sectioned upper arm in wrapping direction of the cuff. The spacer 403a is has a wider separation between core 401a and occlusion component 402a at proximal side than distal side. The width of the spacer can be equal to or smaller than the width of core 401a along the body portion. When the cuff is placed around the body portion, it is preferred that the length of the spacer 403a at the side near to the occlusion component 402a is smaller than the length of the spacer 403a at the side near to the core 401a. 406a shows the projection of the cuff at the opposite side of the cuff for a simple visualization.

Another possibility is shown in FIG. 4-B such that occlusion component 402a in FIG. 4-A is supported by an occlusion support component 406b by a fluidic connection. It is proposed to further suppress the effect of upstreams and to reduce the noise in the oscillation signals. The heart continues to beat, and the pumped blood hits to the wall of the occluded artery 405a and bounces back. These upstreams are noisy and it is the best to reduce the effects of those blood movements. Therefore, a support structure to improve the occlusion at proximal side is proposed.

The third possibility is shown in FIG. 4-C. To improve the fitness or the compliance of the blood pressure cuff further, a compliance fluid bag 408c is employed between occlusion component 402a and body portion. To detect the pulse wave for the estimation of blood pressure value, a pulse-wave detection component 409c is placed between said compliance fluid bag 408c and body portion. An encapsulated compliance fluid bag 402a is preferred. Fluids such as liquids, gels, mixtures with different viscosities are possible. By using such a compliance fluid bag containing liquid or a jelly like high viscosity material, the fitness or the compliance of the cuff is further improved. Spacer improves the fitness, but compliance fluid bag improves the effects further. By utilizing both, fitness to any upper arm of different individuals is possible and arm shape independent cuff is applicable.

Even though it is not shown as figure, it is possible to measure blood pressure without the use of pulse wave detection component 409c. The occlusion component itself can be used both as an occlusion component and as a pulse wave sensing device as usual. Furthermore, it is possible to change the place of the compliance fluid bag such that it can be between the spacer and the occlusion component too.

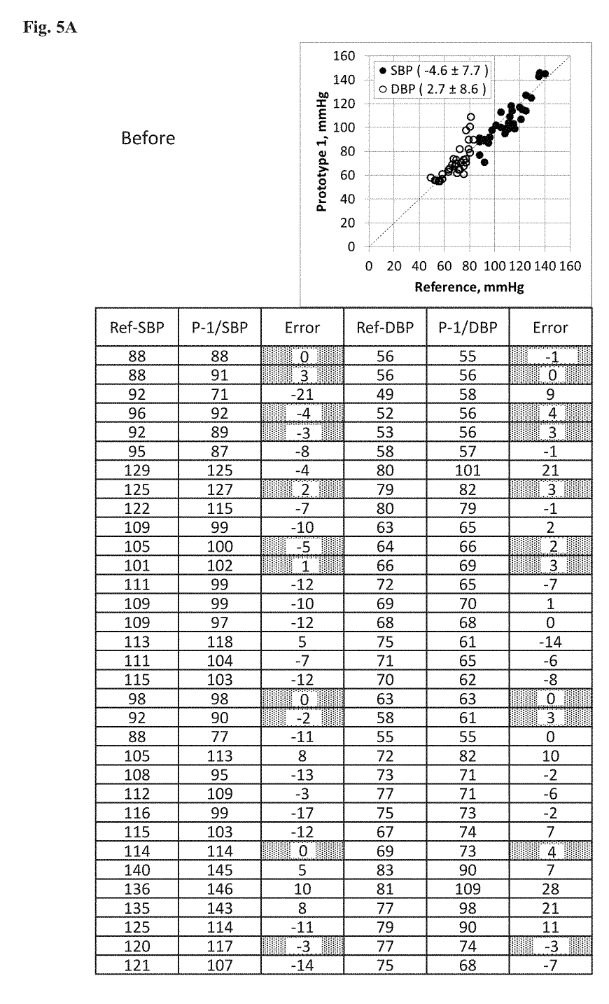

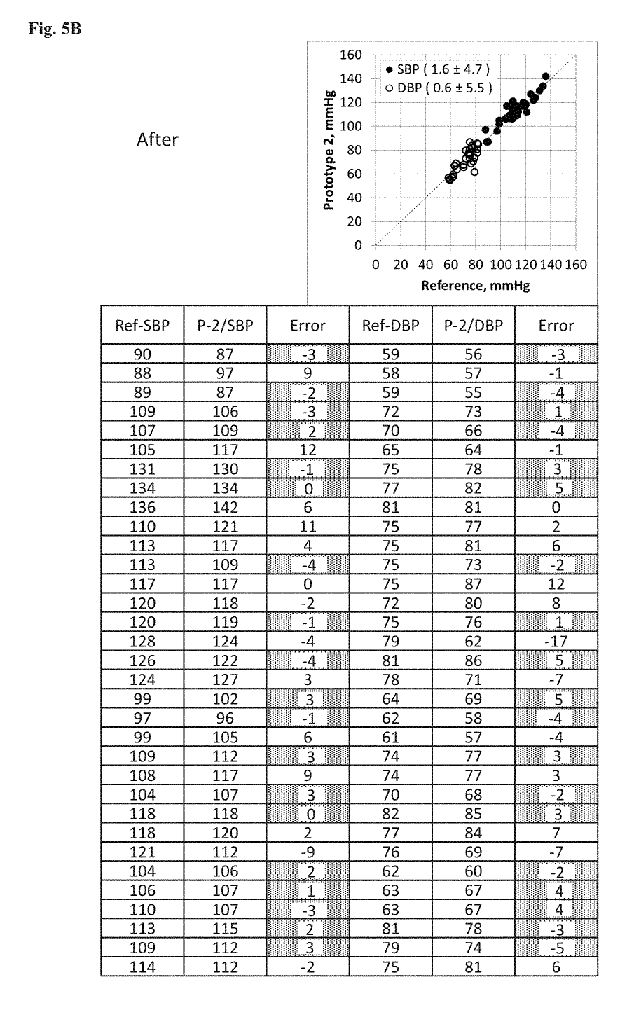

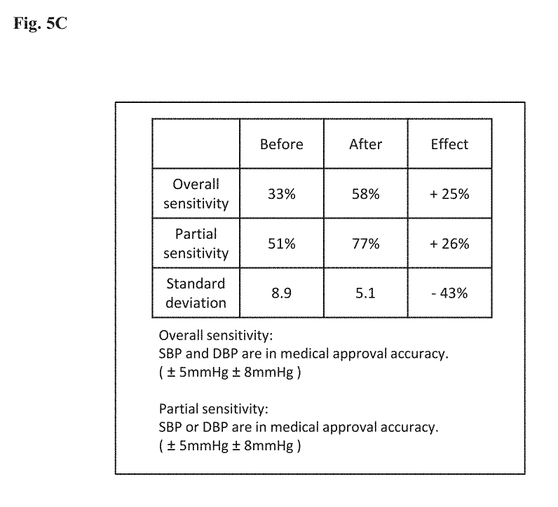

To demonstrate the impact of spacer, experiments are conducted on 11 volunteers with 3 trials (FIGS. 5A-5C, reference device is a commercially available blood pressure meter). In these experiments, compliance fluid bag with jelly like material inside to improve the compliance or the fitness to the body portion is utilized. In the experiments, a cuff structure utilizing a spacer configured to a crescent like structure by using a foamed plastic with enough flexibility and durability are employed. The spacer material is tested at 300 mmHg. (300 mmHg=40 kPa=0.04 MPa, a flexibility bigger than this value is appreciated.) It was flexible but strong and durable enough that no deformation is observed. In before-experiment (prototype 1), the cross-section was similar to FIG. 4-C, but there was no spacer. In after-experiments (prototype 2), spacer is utilized as similar to FIG. 4-C. Before using the spacer, the measured systolic blood pressure (compared to commercial blood pressure) error was −4.6 mmHg in average and 7.7 mmHg in deviation (error). Diastolic blood pressure error was 2.7 mmHg in average and 8.6 mmHg in deviation. These results show that the before-experiments lose the medical accuracy where the limit is 8 mmHg in deviation (error). The device can be sensitive, but when tried on different people, it loses its sensitivity due to differed shaped arms of the individuals.

To increase the accuracy, we employed a spacer and the results are indicated as after-experiments. When spacer is included, systolic blood pressure error was 1.6 mmHg in average and 4.7 mmHg in deviation (error). Diastolic blood pressure error was 0.6 mmHg in average and 5.5 mmHg in deviation.

In both experiments, results within medical approval accuracy (±5±8) are shown in gray boxes to simplify the differences. Overall sensitivity; i.e. SBP and DBP are within medical approval accuracy, is almost improved 25%, while partial sensitivity; i.e. SBP or DBP are within medical approval accuracy, is almost improved 26%. The standard deviation (or error) is reduced almost 43%. The spacer structure in the cuff is very effective.

Furthermore, the errors in average (from −4.6 to 1.6 in SBP, from 2.7 to 0.6 in DBP) get closer to the zero, which is the ideal case. This also shows that spacer is effective and the device is less dependent on arm shapes, and accuracy is further improved.

It is important that the spacer center is roughly positioned around the artery to be measured. It is the best if the spacer is centered on the upper arm artery. The size of the spacer is bigger than the artery size, and therefore the misalignments are tolerable.

This device is smaller than its commercially available counterparts even with half decreased occlusion bag (inflatable), but its medical accuracy is comparable. This makes it attractive in compact blood pressure measurements in daily life or ABPM applications in standard adults.

### Second Exemplary Embodiment

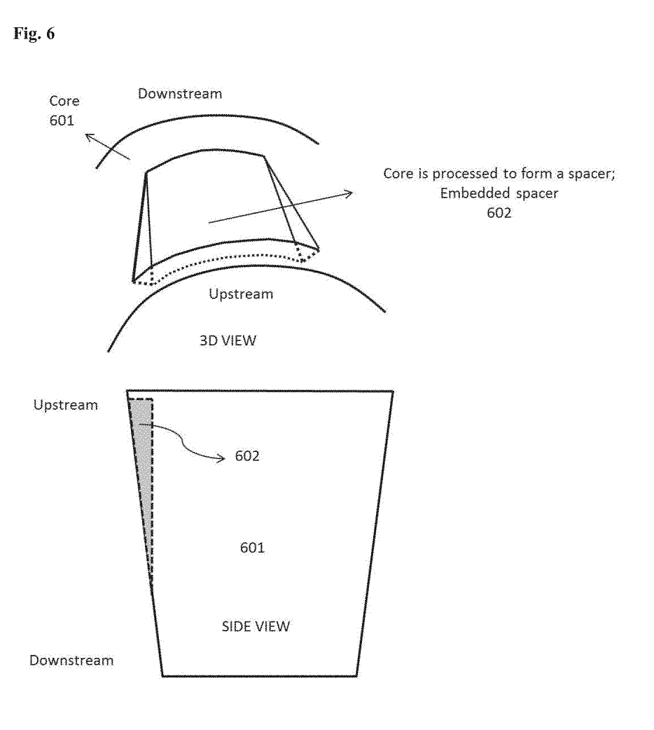

The second exemplary embodiment of the blood pressure cuff is shown in FIG. 6. In this case, the core 601, which is usually plastic material, is processed into a shape on the body portion side. It is such that spacer 302 in FIG. 3 is embedded in FIG. 6 to form embedded spacer 602 in the core 601. The side view shows that the cross section along the body portion is similar to triangle. This means that embedded spacer 602 has deeper at upstream side compared to downstream side (i.e. hand side). The width of the embedded spacer 602 along the body portion can be as wide as the core 601 along that direction. The size of the embedded spacer 602 can be smaller than the size of the core 601 along the wrapping direction.

The advantage of the embedded spacer 602 is that this space or the volume is empty and electrical and electronics circuits, ICs, pumps, valves, or batteries can be positioned in this volume to decrease the thickness of the final blood pressure cuff. Because, the thickness of the cuff in daily life if very effective for portability, wearability and the comfortability.

Embedded spacer 602 provides both improved accuracy and improved wearability. However, the processing a plastic core causes extra cost. It will increase the manufacturing cost.

### Third Exemplary Embodiment

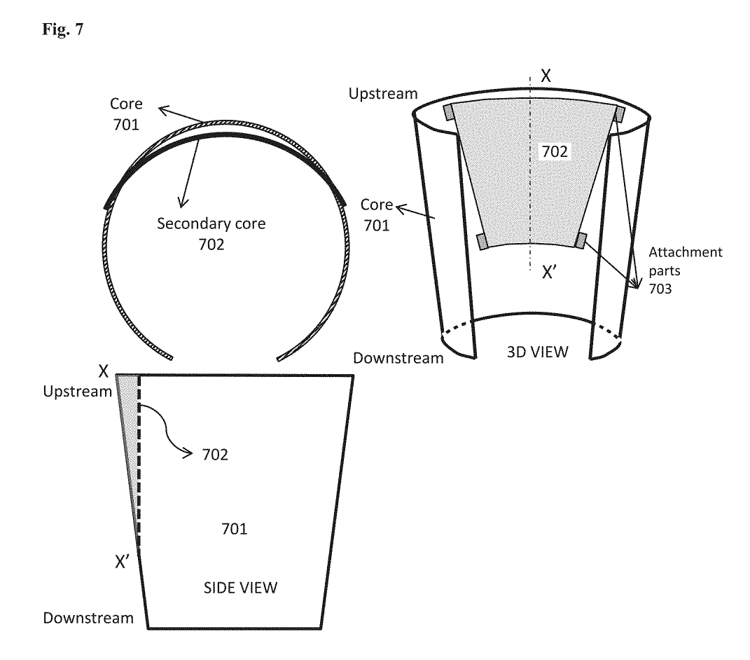

The third exemplary embodiment of the blood pressure cuff is shown in FIG. 7. In this example, a secondary core 702 is attached to the core 701 which is the main core. Attachment can be done by using attachment parts 703. Secondary core 703 can be metal or plastic sheet similar to the core 702. It is such that the separation of secondary core 702 to the core 701 can be bigger at upstream side compared to downstream side.

The width of the secondary core 702 along the body portion can be as wide as the core 701 along that direction. (XX′ in side view can be as long as the width of the core 701.) The size of the secondary core 702 can be equal to or smaller than the size of the core 701 along the wrapping direction.

### Fourth Exemplary Embodiment

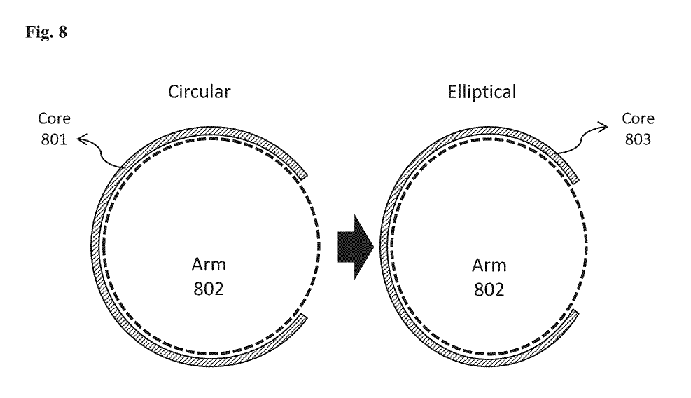

The fourth exemplary embodiment of the blood pressure cuff is shown in FIG. 8. Up to now, circular cross-sections around wrapping direction to the body portion are introduced. Circular core 801 can be attached to a body portion; e.g. arm 802. The core can be configured to be elliptical in cross-section to form core 803. The structure is simple but as mentioned in second exemplary embodiment, processing the core in to a non-standard shape causes extra cost. This will increase the final product cost. Methods are usually preferred with simple approaches without increasing the cost.

In the case of configuring or shaping a material, plastics or metals are appreciated. But, it is placed on a body portion with curvy surfaces, it is better to have high flexibility and enough durability.

### INDUSTRIAL APPLICABILITY

This invention can be applied to the blood pressure meters and ABPMs.

### REFERENCE SIGNS LIST

201, 301, 401a, 401b, 401c, 601, 701, 801, 803, core
202, fastener
203, 404a, 404b, 404c, 802, arm
204, muscle
205, bone
206, artery
207, pressurization volume
208, spacing
210, 303, inner surface
211, 304, outer surface
302, 403a, 403b, 403c, spacer
402a, 402b, 402c, occlusion component
405a, 405b, 405c, occluded artery
406a, 407b, 407c, projections
406b, 406c, occlusion support component
408c, compliance fluid bag
409c, pulse-wave sensing component
602, embedded spacer
702, secondary core

What is claimed is:

1. A blood pressure cuff comprising,
an occlusion component configured to occlude an artery,
a core configured on said occlusion component closer to a body portion to limit the degree of freedom into the body portion,
and
a spacer occupying a volume configured between said occlusion component and said core to enhance a compliance towards body portion.

2. The blood pressure cuff of claim 1,
wherein said spacer is configured to be along the body portion and around a blood vessel to be monitored has a wider spacing between said core and said occlusion component at proximal side of body portion compared to distal side.

3. The blood pressure cuff of claim 1,
wherein said spacer is configured to have equal or smaller length and width compared to the said core.

4. The blood pressure cuff of claim 1,
wherein said spacer is configured to have a cross-section with an inner length near to body portion in wrapping direction to the body portion being smaller than an outer length near to said core when said spacer is in contact with body portion.

5. The blood pressure cuff of claim 1,
wherein said spacer is configured to have an elasticity comparable or less than the elasticity of said core.

6. The blood pressure cuff of claim 1,
wherein said spacer is configured to be an encapsulated fluid bag.

7. The blood pressure cuff of claim 1,
wherein said spacer is configured by processing the said core to embed said spacer,
or,
a secondary core is attached.

8. A blood pressure cuff comprising a core having elliptical cross-section along wrapping direction towards body portion.

9. The blood pressure cuff of claim 1, further comprising:
a compliance fluid bag configured between said occlusion component and body portion to disperse the pressure over the artery,
with/without
a pulse wave detection component configured between said compliance fluid bag and body portion to detect pulse wave or blood pressure.

10. A blood pressure meter wherein a blood pressure cuff in claim 1 is included.

11. A blood pressure meter wherein a blood pressure cuff in claim 8 is included.
